(19) United States
(12) Patent Application Publication
(10) Pub. No.: US 2025/0268875 A1
(43) Pub. Date: Aug. 28, 2025

(54) STABILIZED PHARMACEUTICAL COMPOSITION OF 1,4-DIHYDROPYRIDINES WITH HYDROGEN ATOM TRANSFER (HAT) ANTIOXIDANTS

(71) Applicant: Maiva Pharma Private Limited, Hosur (IN)

(72) Inventors: Alagumurugan Alagarswamy, Hosur (IN); Bhaskar Krishna Arumugam, Bangalore North (IN); Sandeep Sukumar, Bhadravathi (IN)

(21) Appl. No.: 18/973,426

(22) Filed: Dec. 9, 2024

(30) Foreign Application Priority Data
Feb. 28, 2024 (IN) 202441014680

(51) Int. Cl. A61K 31/4422; A61K 47/22; A61K 47/26

(52) U.S. Cl. CPC A61K 31/4422; A61K 47/22; A61K 47/26

(57) ABSTRACT

A stabilized pharmaceutical composition of 1,4-Dihydropyridines (DHP) incorporating hydrogen atom transfer (HAT) antioxidants is disclosed. The composition comprises nicardipine hydrochloride, a pharmaceutically acceptable DHP salt, at concentrations of 0.1 mg/mL to 5 mg/mL. The HAT antioxidants, such as ascorbic acid or Butylated hydroxyanisole (BHA), are added in amounts of 0.001 mg/mL to 25 mg/mL to prevent the sensitization of DHP compounds observed in the commercial production area. Additionally, co-solvents like sorbitol, mannitol or ethanol, ranging from 0.1 to 10 weight percent, enhance solubility. Buffering agents maintain pH between 3 to 5, while tonicity agents adjust osmolarity to 250 mOsm/kg to 350 mOsm/kg. The composition's formulation involves mixing nicardipine hydrochloride with co-solvents, buffering agents, and antioxidants at temperatures of 5° C. to 30° C. Final sterilization is achieved through filtration or terminal moist heat sterilization, ensuring product safety. This pharmaceutical composition offers enhanced stability, solubility, and efficacy, providing a promising treatment option for cardiovascular conditions.

### EARLIEST PRIORITY DATE

This application claims priority from a complete patent application filed in India having patent application No. 202441014680, filed on Feb. 28, 2024, and titled “A STABILIZED PHARMACEUTICAL COMPOSITION OF 1,4-DIHYDROPYRIDINES WITH HYDROGEN ATOM TRANSFER (HAT) ANTIOXIDANTS”.

### TECHNICAL FIELD

The present invention pertains to the field of pharmaceutical compositions, particularly focusing on stabilized formulations of 1,4-dihydropyridines utilizing hydrogen atom transfer (HAT) antioxidants. More particularly, the present invention aims to enhance the stability and shelf life of pharmaceutical compositions containing 1,4-dihydropyridine compounds.

### BACKGROUND OF THE INVENTION

The 1,4-dihydropyridines (“DHPs”), a class of calcium channel blockers are susceptible to oxidative/photolytic degradation of the dihydropyridine ring to the pyridine ring forming pyridine analogues wherein the 1,4-dihydrogens of the dihydropyridine ring are lost by oxidation due to the driving force of becoming a thermodynamically more stable photoproduct, the pyridine analog by aromatization.

For example, Nicardipine pyridine analog is the major oxidative and photolytic degradation impurity reported to be dependent on the pH of the formulation, temperature and availability of air/oxygen.

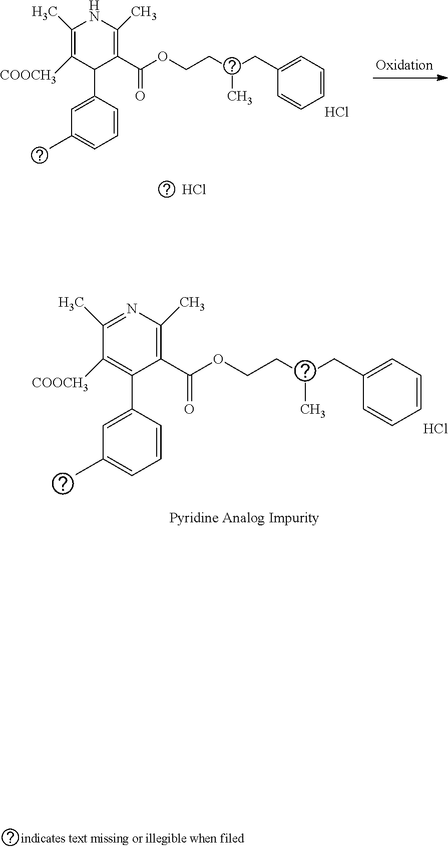 [SMILES: COC(=O)C1=C(C)NC(C)=C(C(=O)OCCC(C)Cc2ccccc2)C1c1cccc(C)c1 ; Cl ; Cl ; COC(=O)c1c(C)nc(C)c(C(=O)OCCC(C)Cc2ccccc2)c1-c1cccc(C)c1 ; Cl]

These pyridine analogues are considered impurities as they possess considerably lower calcium channel blocking activity than their dihydropyridine forms, leading to decreased therapeutic effects and the increase of potential toxicological effects.

Though the DHP Pyridine analog impurity is known to be an oxidative degradant, it is observed that this Pyridine analog impurity does not form significantly in the injectable formulations when manufactured in R&D laboratory conditions without any control on dissolved oxygen or headspace oxygen. However, the DHP Pyridine analog impurity starts forming significantly when the injectable product is manufactured aseptically under production/commercial manufacturing process conditions even though controls are maintained to have minimal dissolved oxygen in the solution and low headspace oxygen in vials. The root cause for this abnormal degradation behavior between the batches manufactured under the R&D lab conditions and commercial production/manufacturing conditions is not clearly understood. However, it appears to be related to the residues of hydrogen peroxide that may remain in the area after the area sanitization or the residues from the sterilization indicators used in the autoclaving of components, leading to the formation of the pyridine analogue degradation impurity.

The RLD, Cardene® 25 mg/10 mL injection has been currently discontinued and there have been multiple product recalls of pharmaceutical products containing the calcium channel blocker nicardipine hydrochloride, mainly related to products that failed impurity specifications. Recently, the limit for Nicardipine pyridine analog impurity in the USP monograph for Nicardipine Injection was increased from NMT 0.9% to NMT 2.5% to accommodate the levels seen in the approved products. Also, various attempts have been reported to stabilize the DHP formulations. For example, U.S. Pat. No. 1,113,520B discloses stabilization of 1,4-dihydropyridine (DHP) compound using chelating agents.

In light of the above, there is a need for pharmaceutical compositions comprising DHPs which are more stable for prolonged periods of time and amenable for commercial production.

### SUMMARY OF THE INVENTION

In the light of the disadvantages mentioned in the previous section, the following summary is provided to facilitate an understanding of some of the innovative features unique to the present invention and is not intended to be a full description. A full appreciation of the various aspects of the invention can be gained by taking the entire specification and drawings as a whole. Embodiments described herein disclose a stabilized pharmaceutical composition of 1,4-Dihydropyridines with hydrogen atom transfer (HAT) antioxidants.

According to an embodiment of the present invention, the stabilized pharmaceutical composition of 1,4-Dihydropyridines with hydrogen atom transfer (HAT) antioxidants comprises a pharmaceutically acceptable salt of 1,4-Dihydropyridines (DHP) comprising nicardipine hydrochloride in an amount of 0.1 mg/mL to 5.0-mg/mL, atleast one hydrogen atom transfer (HAT) antioxidant stabilizer in an amount between 0.001 mg/mL to 25 mg/ml and a quantity sufficient of water to make the formulation to 1 mL.

According to an embodiment of the present invention, wherein hydrogen atom transfer (HAT) antioxidant is selected from hydrophilic compounds such as ascorbic acid, gallic acid, caffeic acid, ferulic acid, coumaric acid and/or lipophilic compounds such as Butylated hydroxyanisole (BHA), Butylated hydroxytoluene (BHT), or derivatives thereof.

According to an embodiment of the present invention, the composition further comprises a tonicity agent selected from sodium chloride, dextrose, sucrose, xylitol, fructose, glycerol, sorbitol, mannitol to obtain a tonicity between 250 mOsm/kg to 350 mOsm/kg.

According to an embodiment of the present invention, the composition may further comprise a co-solvents selected from sorbitol, mannitol, xylitol, propylene glycol, polyethylene glycol, ethanol or a combination thereof, in an amount of 1 mg/mL to 50 mg/mL of the composition. Some of the co-solvents may also function as tonicity adjusting agents.

According to an embodiment of the present invention, the compositions may further comprise a buffering agent selected from citrate, acetate, glutamate, tartrate, benzoate, lactate, histidine, amino acids, gluconate, phosphate, succinate, or a combination thereof, in an amount of 0.1 mg/mL to 50 mg/mL weight percent of the composition. The pH of the formulation is adjusted between 3 to 5 using pH adjusting agents such as hydrochloric acid and/or sodium hydroxide, if necessary.

According to an embodiment of the present invention, wherein the composition is prepared by forming a aqueous solution of nicardipine hydrochloride with hydrogen atom transfer (HAT) antioxidant(s) and co-solvents, tonicity adjusting agents, buffering agent as necessary, wherein the pH of the formulation is adjusted between 3 to 5. Further, sterilizing the prepared composition by filtering through one or more filters with a pore size of 0.22 μm along with aseptic processing, or by terminal moist heat sterilization or combinations thereof.

According to an embodiment of the present invention, the composition further comprises a tonicity agent selected from sodium chloride, dextrose, sucrose, xylitol, fructose, glycerol, sorbitol, mannitol, or sodium chloride to maintain an osmolality between 250 mOsm/kg to 350 mOsm/kg.

According to an embodiment of the present invention, a pharmaceutical composition comprises a 1,4-dihydropyridine compound comprising nicardipine in an amount of 2.5 mg/mL of the composition and atleast one antioxidant acting by Hydrogen atom transfer (HAT) in an amount of 0.001 mg/mL to 25 mg/mL, wherein the HAT antioxidant is preferably Ascorbic acid or its pharmaceutically acceptable salts thereof.

According to an embodiment of the present invention, a stabilized pharmaceutical composition comprising nicardipine hydrochloride in an amount of 1 mg/mL to 10 mg/mL, a co-solvent comprising sorbitol in an amount that is 40 mg/mL to 55 mg/ml of the composition, a buffering agent comprising citrate buffer in an amount between that is 0.1 mg/mL to 5 mg/mL of the composition, and atleast one antioxidant acting by Hydrogen atom transfer (HAT) in an amount of 0.001 mg/mL to 25 mg/mL, wherein the HAT antioxidant is preferably Ascorbic acid or its pharmaceutically acceptable salts thereof, wherein the stabilized composition comprises a pyridine analog impurity in an amount less than 1% w/w of the DHP compound.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE ACCOMPANYING DRAWINGS

The detailed description is provided with reference to the accompanying FIGURES. These and other features and advantages of the present invention will be readily appreciated, as the same becomes better understood by reference to the following detailed description when considered in connection with the accompanying drawings wherein:

FIG. 1 illustrates a method for preparing a stabilized pharmaceutical composition of 1,4-dihydropyridines with hydrogen atom transfer (HAT) antioxidants in accordance with an embodiment of the present invention.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures, or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Compounds for use in the compositions described herein that may contain one or more asymmetric centers can thus occur as racemates and racemic mixtures, single enantiomers. The compositions are meant to comprehend all isomeric forms of such compounds.

The term “calcium channel blocker(s)” refers to compounds that inhibit, reduce or prevent the movement of calcium into and/or within cells.

The term “composition(s)” refers to an aggregate material formed from two or more substances, ingredients or constituents; the way in which a whole or mixture is made up. When referring to pharmaceutical drug products, a composition is often called “formulation(s)”.

The term “dihydropyridine(s)” refers to molecules based upon pyridine, and the parent of a class of molecules that have been semi-saturated with two substituents replacing one double bond.

The term “dosage form(s)” refers to pharmaceutical drug products in the form in which they are marketed for use, administration or consumption, with a specific mixture of active ingredient(s) and inactive component(s) (excipients), in a particular configuration. Examples of dosage forms include without limitation, liquid, solid, semisolid, suspension, powder, crystal, paste, oral (pill, tablet, capsule, film, solution, liquid, syrup, buccal), inhalational, device (aerosol, inhaler, nebulizer, vaporizer, pen), sublingual, nasal, suppository (vaginal, rectal, urethral, nasal), parenteral (intradermal, subcutaneous, intramuscular, intraosseous, intraperitoneal, intravenous), topical (cream, gel, liniment, balm, lotion, ointment, drop, patch, ring, talc), ophthalmic and injectable.

The term “impurity” refers to a constituent, component or ingredient which impairs the purity of pharmaceutical active ingredient or pharmaceutical composition.

The term “injectable” or “injectable composition,” as used herein, means a composition that can be drawn into or filled in a container and injected intravenously, subcutaneously, intramuscularly, intra-arterially, intra-cardiac, intrathecally, epidurally, intraparenchymally, intraperitoneally, intracerebroventricularly, intraventricularly, or the like into an animal.

The term “reference listed drug” refer to an approved drug product to which generic versions are compared to show that they are bioequivalent.

The term “stability” refers to capability of a pharmaceutical active ingredient or pharmaceutical composition to remain within a specific criteria or specification(s).

The term “stable”, as used herein, means remaining in a state or condition that is suitable for administration to a patient and without undergoing a substantial change in the potency of the active agent in the formulation over the specified time period. In some embodiments, compositions are stable when maintained at room temperature for at least 6 months, usually at least 12 months, and generally for at least 18, 24, 36 or 48 months. In some embodiments, the compositions are also preferably stable over more extended periods of time when stored at 25° C. A substantial change in potency is one which decreases the drug concentration by more than 10% from the target concentration for the specified period of time. Unless indicated otherwise, a stable composition is one which retains at least 90% of the original amount of the 1,4-dihydropyridine compound in that state (e.g., not precipitated, degraded or adsorbed to the container) for a period of at least six months at the appropriate storage conditions.

The carriers and excipients and other components of the pharmaceutical compositions must be “pharmaceutically acceptable” in the sense of being compatible with the other ingredients of the formulation and not deleterious to the recipient thereof. Thus, the term “pharmaceutically acceptable salt” references salt forms of the active compounds which are prepared with counter ions which are non-toxic under the conditions of use and are compatible with a stable formulation. For compounds which contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. Salts derived from pharmaceutically acceptable organic nontoxic bases include salts of primary, secondary, and tertiary amines, substituted amines including naturally occurring substituted amines, cyclic amines, ethanolamine, 2-diethylaminoethanol, lysine, arginine, and histidine.

For compounds which contain relatively basic functionalities, acid addition salts can be obtained by contacting the neutral form of such compounds with a sufficient amount of the desired base, either neat or in a suitable inert solvent. Examples of “pharmaceutically acceptable acid addition salts” (i.e., salts retaining the biological effectiveness and properties of the free bases and which are not biologically or otherwise undesirable), can be formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

The term “pharmaceutically acceptable carrier or excipient” means a carrier or excipient that is useful in preparing a pharmaceutical composition that has an acceptable side-effect profile and serves to provide a medium for the storage or administration of the active component(s) under the conditions of administration for which the composition is formulated or used. The carrier or excipient is compatible with the other ingredients of the formulation and not deleterious to the recipient thereof. A “pharmaceutically acceptable carrier or excipient” as used in the specification and claims includes both one and more than one such carrier or excipient. Pharmaceutically acceptable carriers are determined in part by the particular composition being administered, as well as by the particular method used to administer the composition. Also, an excipient can perform more than one function. For example, sorbitol can act as both a tonicity agent and a co-solvent. There are a wide variety of suitable formulations of pharmaceutical compositions of the present disclosure (see, e.g., Remington's Pharmaceutical Sciences, 20th ed., 2018, supra).

The term “antioxidant” refers to a substance that inhibits or delays oxidation. Antioxidant compounds act through several chemical mechanisms: hydrogen atom transfer (HAT), single electron transfer (SET), and the ability to chelate transition metals. In the HAT mechanism, radicals are quenched by hydrogen atom donation of the antioxidant and in the single electron transfer (SET) mechanism, the antioxidant's ability to transfer one electron and thus, reduce the other compound is used.

The HAT antioxidants refer to antioxidants that directly donates hydrogen ion from a stable molecule thus allowing the antioxidant to scavenge the reactive oxygen species. Further, the antioxidants acting by hydrogen atom transfer (HAT) could be hydrophilic or lipophilic in nature. Suitable hydrophilic or water-soluble HAT antioxidants include ascorbic acid, gallic acid, caffeic acid, ferulic acid, coumaric acid, or its derivatives. Suitable lipophilic HAT antioxidants include Butylated hydroxy toluene (BHA), Butylated hydroxy toluene (BHT) or its derivatives.

The HAT antioxidant, ascorbic acid or its derivatives such as sodium ascorbate are preferred due to its common use in injectable preparations and aqueous solubility. Ascorbic acid is a donor of single reducing equivalents and the radical it forms, monodehydroascorbate reacts preferentially with radicals instead of with non-radical compounds to form dehydroascorbate.

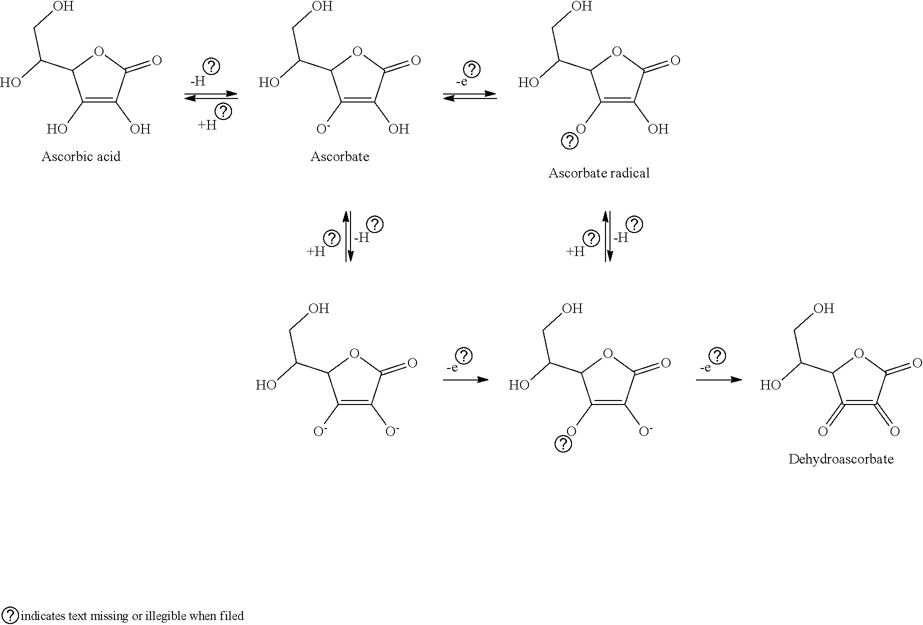

Reference: David Njus et. al., Free Radic Biol Med. 2020 Nov. 1; 159:37-43. doi: 10.1016/j.freeradbiomed.2020.07.013

Alternatively, lipophilic HAT antioxidants, such as Butylated hydroxy toluene (BHA), Butylated hydroxy toluene (BHT) could be used appropriately.

The term “tonicity adjusting agents” refers to agents used to modify the osmolality of a formulation to bring it closer to the osmotic pressure of body fluids such as blood or plasma. Provided that the compositions are physiologically compatible, the compositions do not require any osmolality. Thus, the compositions can be hypotonic, isotonic, or hypertonic. Typically, the pharmaceutical compositions have an osmolality between about 250 to 350 mOsm/kg. The tonicity of the pharmaceutical compositions can be adjusted by adjusting the concentration of any one or more of a tonicity agent, a co-solvent, buffering agent, or excipient. Suitable tonicity adjusting agents include, but are not limited to, anhydrous and hydrous forms of dextrose, sucrose, xylitol, fructose, glycerol, sorbitol, mannitol and sodium chloride, propylene glycol etc.

“Buffering agents” are agents used to control the pH of a formulation. A variety of buffering agents are suitable and may be used alone or together in the composition. Suitable buffering agents include, but are not limited to, acids and salts of acetate, benzoate, glutamate, citrate, tartrate, benzoate, lactate, amino acids, gluconate, succinate, MES, phosphate or a combination thereof. In some embodiments, the buffering agent can function as a tonicity agent.

The pH of the pharmaceutical composition can be adjusted to the recited pH range or target pH by the addition of an acid or acidic salt (e.g., HCl, phosphoric acid), or base or basic salt, as appropriate. For instance, the pH may be adjusted with a base such as an alkali metal hydroxide such as NaOH. The buffering agent may be the acid or base form of the species which gives rise to the salt of the nicardipine.

A “co-solvent” is a solvent which is added to the formulation and assists in the solubilization of the 1,4-dihydropyridine compound(s). Co-solvents, for instance, can be selected from the group consisting of polyhydric alcohols (e.g., sorbitol, mannitol, xylitol), glycols (e.g., propylene glycol and polyethylene glycol), organic liquids (e.g., ethanol), or water. Typically, the co-solvent is in a concentration from 0.1% to 10%.

The term “pharmaceutical composition” is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients described herein. In one embodiment, a pharmaceutical composition generally comprises a therapeutically effective amount of nicardipine, one or more buffering agents, and other ingredients as described herein.

The term “therapeutically effective amount” refers to an amount of an agent sufficient to treat or prevent acute elevations in blood pressure or induce hypotension when administered alone or as one of multiple dosages to a subject. The “therapeutically effective amount” will vary depending on the formulation, the severity of the blood pressure elevation, the age, general health condition, and weight of the subject to be treated.

The term “pre-mixed”, as used herein, means a pharmaceutical composition that is already mixed from the point of pre-sale packaging and/or manufacture and does not require reconstitution or dilution before administration to a subject.

The term “single-use container” refers to a sealed pharmaceutically prepared container holding a drug product in a sterile environment that is intended to be used in a single operation of transferring the entire contents or substantially entire contents. It should be recognized that the single-use container is generally preservative-free.

As used herein, the term “mixing” refers to admixing, contacting, blending, stirring or allowing to admix, mix, blend, stir and the like.

The term “dissolved oxygen” refers to oxygen that is found in the aqueous carrier of the compositions. Distinguished from dissolved oxygen is the headspace oxygen. As used herein, the term “headspace oxygen” refers to the oxygen that is found in the headspace volume of the sealed container comprising the composition.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

Embodiments described herein disclose a stabilized pharmaceutical composition of 1,4-dihydropyridines (DHP) with hydrogen atom transfer (HAT) antioxidants.

According to an embodiment of the present invention, the stabilized pharmaceutical composition of 1,4-Dihydropyridines with hydrogen atom transfer (HAT) antioxidants comprises a pharmaceutically acceptable salt of 1,4-Dihydropyridines (DHP) comprising nicardipine hydrochloride in an amount of 0.1 mg/mL to 5.0 mg/mL, atleast one hydrogen atom transfer (HAT) antioxidant stabilizer in an amount between 0.0001 mg/mL to 25 mg/mL.

According to an embodiment of the present invention, wherein the at least of hydrogen atom transfer (HAT) antioxidant is selected from hydrophilic or lipophilic compounds comprising ascorbic acid, gallic acid, caffeic acid, ferulic acid, coumaric acid, Butylated hydroxyanisole (BHA), Butylated hydroxytoluene (BHT), or derivatives thereof.

According to an embodiment of the present invention, the composition further comprises a co-solvent selected from sorbitol, mannitol, xylitol, propylene glycol, polyethylene glycol, ethanol, water, or a combination thereof, in an amount of 0.1 mg/mL to 50 mg/mL of the composition.

According to an embodiment of the present invention, wherein the stabilized composition comprises a pyridine analog impurity in an amount less than 1 weight percent of the composition.

According to an embodiment of the present invention, the composition further comprises a buffering agent selected from citric acid, anhydrous citric acid, citric acid monohydrate, sodium hydroxide, acetate, glutamate, citrate, tartrate, benzoate, lactate, histidine, amino acids, gluconate, phosphate, succinate, or a combination thereof, in an amount of 0.1 mg/mL to 50 mg/mL of the composition. The pH of the formulation is adjusted between 3 to 5 using pH adjusting agents such as hydrochloric acid and/or sodium hydroxide, if necessary.

According to an embodiment of the present invention, the composition further comprises a tonicity agent selected from sodium chloride, dextrose, sucrose, xylitol, fructose, glycerol, sorbitol, mannitol, or sodium chloride at a suitable concentration to maintain osmolality between 250 mOsm/kg to 350 mOsm/kg.

According to an embodiment of the present invention, the composition further comprises a tonicity agent selected from sodium chloride, dextrose, sucrose, xylitol, fructose, glycerol, sorbitol, mannitol, or sodium chloride to maintain an osmolality between 250 mOsm/kg to 350 mOsm/kg.

According to an embodiment of the present invention, a pharmaceutical composition comprises a 1,4-dihydropyridine compound comprising nicardipine in an amount of 2.5 mg/mL of the composition and atleast one antioxidant acting by Hydrogen atom transfer (HAT) in an amount of 0.001 mg/mL to 25 mg/mL, wherein the HAT antioxidant is preferably Ascorbic acid or its pharmaceutically acceptable salts thereof.

According to an embodiment of the present invention, a stabilized pharmaceutical composition comprising nicardipine hydrochloride in an amount of 1 mg/mL to 10 mg/mL, a co-solvent comprising sorbitol in an amount that is 40 mg/mL to 55 mg/ml of the composition, a buffering agent comprising citrate buffer in an amount between that is 0.1 mg/mL to 5 mg/mL of the composition, and atleast one antioxidant acting by Hydrogen atom transfer (HAT) in an amount of 0.001 mg/mL to 25 mg/mL, wherein the HAT antioxidant is preferably Ascorbic acid or its pharmaceutically acceptable salts thereof, wherein the stabilized composition comprises a pyridine analog impurity in an amount less than 1% w/w of the DHP compound.

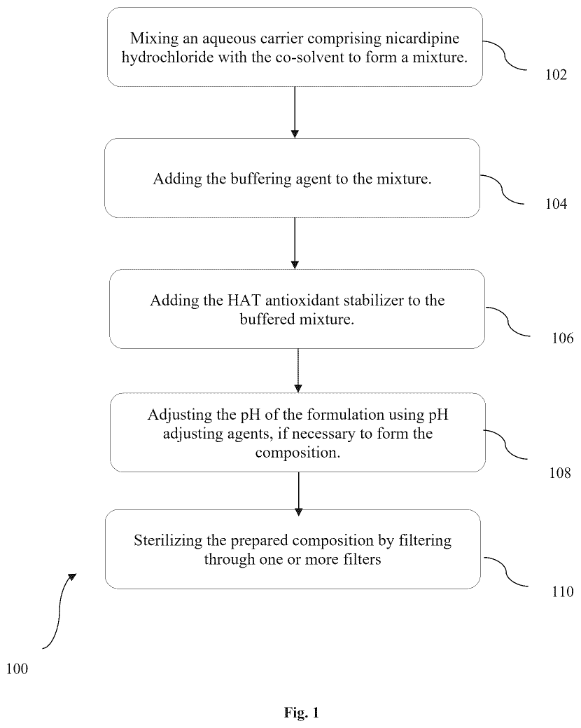

FIG. 1 illustrates a method 100 for preparing a stabilized pharmaceutical composition of 1,4-dihydropyridines with hydrogen atom transfer (HAT) antioxidants in accordance with an embodiment of the present invention. According to an embodiment of the present invention, wherein the composition is prepared by mixing an aqueous carrier comprising nicardipine hydrochloride with the co-solvent to form a mixture (step 102), then adding buffering agents to form a solution (step 104) and adding the HAT antioxidant stabilizer to the buffered mixture to form the composition (step 106) and then finally adjusting the pH using the pH adjusting agents, if necessary (Step 108), wherein the mixing is carried out at temperatures ranging from about 15° C. to about 30° C. Further, sterilizing the prepared composition by filtering through one or more filters (step 110) with a pore size of 0.22 μm and aseptic processing, or by terminal moist heat sterilization or combinations thereof.

In various embodiments, the present disclosure relates to pre-mixed, ready-to-use, injectable pharmaceutical compositions comprising a DHP or a pharmaceutically acceptable salt, hydrate or solvate thereof and at least one HAT antioxidant in aqueous medium. The composition may further contain a solvent, co-solvent, and buffering agent. The composition may further comprise a tonicity agent. The compositions are preferably isotonic.

According to an embodiment of the present invention, the pharmaceutical compositions of the present application can be made by mixing an aqueous carrier, such as for example, water with a co-solvent and/or a tonicity adjusting agent as discussed above to form a mixture. To the mixture, a buffering agent can be added to form a buffered mixture. To the buffered mixture the 1,4-dihydropyridine compound and a water-soluble HAT antioxidant stabilizer can be added to form the pharmaceutical composition. The mixing can be done at temperatures from about 5° C. to about 50° C., for example, at 15° C. to 30° C. If necessary, the pH can be adjusted further using an acid or a base.

### WORKING EXAMPLES

DHP (1,4-dihydropyridines) Compositions

The disclosure described herein relates to the development of DHP compositions or formulations in which the inclusion of an antioxidant stabilizer acting by Hydrogen atom transfer results in more stable compositions.

More specifically, the present disclosure is directed to pharmaceutical compositions having at least one DHP compound and at least one antioxidant stabilizer acting by Hydrogen atom transfer. The DHP compound, preferably, one or more of nicardipine, nifedipine, nimodipine, clevidipine, amlodipine, isradipine, felodipine, nisoldipine, azelnidipine, cilnidipine, nitrendipine, aranidipine, barnidipine, benidipine, efonidipine lacidipine, lercanidipine, manidipine, nilvadipine, nitrendipine, or free bases, hydrates, solvates, crystalline polymorphs, amorphous forms, or pharmaceutically acceptable salts thereof.

The compositions can be administered by parenteral routes, including, subcutaneous, intramuscular, and intravenous routes, to a patient. The compositions can be provided in a single-use vial for injection, a multidose vial for injection or a premixed intravenous container suitable for an intravenous infusion.

In certain embodiments, pharmaceutical compositions of this disclosure relate to nicardipine composition for injection as a direct bolus intravenous injection or to be diluted in a suitable delivery vehicle containing dextrose, saline or a combination thereof and administered to a human subject as a bolus dose or intravenous infusion. Further, when referring to the DHP compound or particularly nicardipine, the active ingredient may not only be in the salt form, but also in the base form (e.g., free base).

In one embodiment, the pharmaceutical composition comprises 0.1 mg/mL to 5.0 mg/mL, inclusive of nicardipine (as calculated for either nicardipine base or its hydrochloride salt) in an aqueous formulation having one or more buffering agent(s) each in a concentration from 0.1 mM to 50 mM, and a pH from about 3.0 to 5.0, inclusive, and one or more additional pharmaceutically acceptable excipients or carriers. In some embodiments, the buffering agent(s) can be any one or more salts and acids of citrate, malate, formate, succinate, acetate, benzoate, propionate, histidine, carbonate, tartrate or phosphate.

Optionally, the compositions comprise a tonicity adjusting agent and/or a co-solvent. Suitable tonicity adjusting agents include but are not limited to, dextrose, sucrose, sorbitol or sodium chloride. Suitable co-solvents include but are not limited to, polyhydric alcohols (e.g., sorbitol, mannitol, xylitol, glucose), glycols (e.g., propylene glycol and polyethylene glycol), and ethanol in a concentration range varying from 0.1 to 15% w/v. Typically, the compositions are provided as low volume, pre-mixed, ready-to-use, bolus injectable, aqueous pharmaceutical compositions or they can be further diluted and administered as an intravenous infusion.

Examples of pharmaceutically acceptable salts of DHP (e.g., nicardipine) are hydrochlorides, sulfates, phosphates, acetates, fumarates, maleates and tartrates. In some embodiments, the pharmaceutically acceptable salt of nicardipine is nicardipine hydrochloride.

The DHP can be made in a solution at the appropriate concentration suitable for injection and be in a solution having a pH range between 3.0 and 5.0. DHP composition can have, suitable buffers, tonicity adjusting agents, pH adjusting agents, solvents, co-solvents and atleast one water soluble antioxidant stabilizer to make it suitable for injection.

In various embodiments, the DHP compositions (e.g., nicardipine) described in this disclosure are in an aqueous formulation having one or more buffering agent(s) each in a concentration from 0.1 mM to 100 mM, and a pH from about 3.0 to 5.0, inclusive. For example, in some embodiments, the composition comprises a buffering agent from 0.5 mM to 50 mM in concentration and a pH more than 3.0 but less than 5.0. In some embodiments, the buffering agent(s) can be any one or more of an acid or salt of citrate, malate, formate, succinate, acetate, benzoate, propionate, histidine, carbonate, tartrate. phosphate, or MES.

In some embodiments, the buffer is a single buffer selected from the group comprising acetate, citrate, succinate, and phosphate. In some embodiments, the compositions comprise two, three, four, or more different buffering agents. For example, in some embodiments, the compositions comprise two buffering agents, selected from the group consisting of acetate and citrate; acetate and phosphate; acetate and succinate; citrate and phosphate; citrate and succinate; and succinate and phosphate. In other embodiments, the compositions comprise three or more buffering agents selected from the group consisting of acetate, phosphate and succinate; citrate, phosphate and acetate; succinate, phosphate and citrate; and citrate, acetate and succinate buffering agents. In some embodiments, the buffering agent can be in powder form and added to the DHP composition to form the injectable DHP composition.

Optionally, the DHP compositions can comprise tonicity adjusting agents and/or co-solvents. In some embodiments, the tonicity adjusting agent is dextrose or sodium chloride. In some embodiments, the co-solvent is polyhydric alcohols (e.g., sorbitol, mannitol, xylitol), glycols (e.g., propylene glycol and polyethylene glycol), and ethanol, in a concentration range varying from 0.1 to 15% w/v.

Typically, the compositions are provided as low volume, pre-mixed, ready-to-use, injectable, bolus aqueous pharmaceutical compositions. In certain aspects, the injection composition of the present invention may be available as a concentrate composition to be mixed with infusion fluids such as 0.9% sodium chloride or 5% dextrose before administration to the patient, or ready to use compositions which are premixed with infusion fluids such as 0.9% sodium chloride or 5% dextrose.

In some embodiments, the formulations comprise (a) nicardipine or a pharmaceutically acceptable salt thereof, (e.g., nicardipine hydrochloride), (b) at least one antioxidant stabilizer acting by Hydrogen atom transfer; (c) a tonicity agent; (d) optionally a co-solvent; and (e) optionally a buffering agent in which the pH of the composition is maintained between 3.0 and 5.0.

In one embodiment, the DHP is nicardipine hydrochloride in a 2.5 mg/mL solution concentration. In another embodiment, the DHP is nicardipine hydrochloride in a 0.1 mg/mL or 0.2 mg/mL solution for intravenous administration in either dextrose or sodium chloride solution and an antioxidant stabilizer acting by Hydrogen atom transfer (HAT).

pH adjusting agents such as Hydrochloric acid and/or sodium hydroxide may be added to adjust pH to 3.0 to 5.0 of the pharmaceutical composition, if necessary.

### Methods of Making and Use

According to the embodiments of the present disclosure, the components of the pharmaceutical composition (e.g., DHP compound, HAT antioxidant stabilizer, solvent, co-solvent, buffering agent, pH adjusting agents etc.) can be mixed in any order. After, mixing and the additions of the components, the pharmaceutical composition can be sterilized, for example, by filtering it through one or more filters (e.g., 0.22 p.m sterile filters). The sterilized pharmaceutical composition can then be filled in the appropriate container (e.g., vial, ampule or bag etc.) and stoppered and sealed, for example, under a reduced oxygen headspace of either 5% oxygen (balance nitrogen) or 10% oxygen (balance nitrogen). In some embodiments, the pharmaceutical composition can be packaged in a pharmaceutically acceptable container, such as an intravenous bag, syringe, vial or ampule. The pH of the compositions is, in some aspects, between 3 and 5, and in other aspects, from 3.0 to 4.5.

The present disclosure also relates to methods for preparing such compositions. In this other aspect, the term “pre-mixed”, as used herein, means a pharmaceutical composition that is already mixed from the point of manufacture and does not require dilution or further processing before administration.

In various embodiments, pre-mixed injection can be a single-use, ready-to-use, iso-osmotic solution for intravenous administration. No further dilution would be required. Prior to administration, the pre-mixed injection should be visually inspected for particulate matter and discoloration prior to administration, whenever solution and container permit. A pre-mixed injection is normally a clear, colorless to yellow solution.

In many aspects, the pharmaceutical compositions are aqueous solutions that are administered by injection. Preferably, the compositions are used for the treatment of cardiovascular and cerebrovascular conditions. In one embodiment, the pharmaceutical compositions of the present disclosure comprise a cardiac medication or a pharmaceutically acceptable salt thereof. In certain embodiments, the cardiac medication is a calcium channel antagonist or a pharmaceutically acceptable salt thereof. For example, the cardiac medication is a dihydropyridine derivative or a pharmaceutically acceptable salt thereof. In various embodiments, the cardiac medication is nicardipine or a pharmaceutically acceptable salt thereof.

According to an embodiment of the present invention, the pharmaceutical compositions comprise from 0.1 to 15 mg/mL nicardipine or a pharmaceutically acceptable salt thereof. For example, suitable concentrations of nicardipine or a pharmaceutically acceptable salt thereof, include, but are not limited to: 0.1 mg/mL, 0.2 mg/mL or 2.5 mg/mL.

Therapeutically effective amounts include dosage ranges of the DHP (e.g., nicardipine) to achieve the desired effect. In some embodiments, the dosage range for nicardipine can be from 10 mg to 120 mg per day or higher.

In some embodiments, a pharmaceutical composition of nicardipine hydrochloride is administered as an I.V. infusion of 20 mg in 200 mL (0.1 mg/mL) at an initial rate of 25 mL (2.5 mg) every 5 to 15 minutes, or 50 mL/hr (5 mg/hr). If the desired blood pressure reduction is not achieved at this dose, the infusion rate may be increased by 25 mL/hr (2.5 mg/hr) every 5 minutes (for rapid titration) to 15 minutes (for gradual titration) up to a maximum of 150 mL/hr (15 mg/hr), until desired blood pressure reduction is achieved. In other embodiments, following the achievement of the blood pressure goal utilizing rapid titration, the infusion rate can be decreased to 30 mL/hr (3 mg/hr).

In other embodiments, a pharmaceutical composition of nicardipine hydrochloride is administered as an I.V. infusion 40 mg in 200 mL (0.2 mg/mL) at an initial rate of 25 mL/hr (5 mg/hr). If the desired blood pressure reduction is not achieved at this dose, the infusion rate may be increased by 12.5 mL/hr (2.5 mg/hr) every 5 minutes (for rapid titration) to 15 minutes (for gradual titration) up to a maximum of 75 mL/hr (15 mg/hr), until desired blood pressure reduction is achieved. Following the achievement of the blood pressure goal utilizing rapid titration, the infusion rate can be decreased to 15 mL/hr (3 mg/hr).

In another aspect, the pharmaceutical compositions can be used to treat cardiac conditions. In many embodiments, the compositions can be used to treat conditions that are alleviated by the administration of calcium channel antagonists, such as cardiovascular and cerebrovascular conditions. Cardiovascular conditions that can be treated with the pharmaceutical compositions of the present disclosure include angina, ischemic, systemic arterial hypertension, congestive heart failure, coronary artery disease, myocardial infarction, cardiac arrhythmias, cardiomyopathies and arteriosclerosis. Cerebrovascular conditions that can be treated with the pharmaceutical compositions of the present disclosure include pulmonary hypertension, cerebral insufficiency, acute cerebral hemorrhage, and migraine. In some embodiments, the compositions are used to treat hypertension.

In certain aspects, the pharmaceutical compositions of the present disclosure also comprise at least one co-solvent. Therefore, the compositions may comprise a co-solvent, or multiple co-solvents.

In another aspect, nicardipine and its pharmaceutically acceptable salts are only slightly soluble in water. Co-solvents can help solubilize nicardipine in the aqueous solution of the pharmaceutical composition. Co-solvents are especially beneficial when a high concentration of nicardipine is present, such as in the compositions of the present disclosure. An advantage of the compositions of the present disclosure is that they have a high concentration of nicardipine, which allows the composition to be administered using a lower volume of intravenous fluid. Such compositions can be a treatment option for a greater number of patients, especially volume restricted patients.

In another embodiment, patients and medical conditions that may benefit from a higher concentration and lower fluid volume of nicardipine include, but are not limited to, the following: acute congestive cardiac failure; pediatrics; hypertensive crises in elderly patients where fluid overload is a major concern; all acute stroke areas including AIS, ICH and SAH to control blood pressure; controlled hypotension during surgical procedures including cardiothoracic surgery (CABG, coarctation of the aorta, etc.), spinal surgeries, and head and neck surgeries; and neurosurgery for the control of breakthrough hypertension post carotid endarterectomy, traumatic brain injury and potential treatment of hypertension and vasospasm.

Furthermore, changes may be made to the concentration of co-solvents, buffering agents and water soluble HAT antioxidants in the pharmaceutical compositions in order to adjust the tonicity of the pharmaceutical compositions. Pharmaceutically acceptable co-solvents are known in the art and are commercially available. Useful co-solvents include, for example, polyethylene glycol (PEG), propylene glycol (PG), ethanol, sorbitol.

In some aspects, the co-solvent concentration is 0.1-10% weight/volume percent. In other aspects, the co-solvent concentration is 0.1-5%. In various embodiments, co-solvents for the pharmaceutical compositions are propylene glycol and sorbitol. In certain aspects, the concentration of propylene glycol is 0.1 to 3%. In other aspects, the concentration of sorbitol is 0.1 to 5%.

In addition, the pharmaceutical compositions of this disclosure can comprise a buffering agent and, in some embodiments, the compositions may comprise multiple buffering agents. The pharmaceutical compositions of the present disclosure are preferably close to physiological pH in order to minimize the incidence of phlebitis upon administration. However, the pH of the pharmaceutical composition also affects the solubility and stability of nicardipine in the composition. Generally, as the pH of the pharmaceutical composition increases, the aqueous solubility of nicardipine decreases. As a result, it is difficult to solubilize nicardipine close to a physiological pH. In addition, the composition should have sufficient buffering capacity such that the solution does not precipitate upon dilution with blood when administered.

Buffering agents are used to adjust the pH of the pharmaceutical compositions in this alternative aspect as well. The pH of the compositions is, in some cases, between 3.0 and 5.0. Most preferably, the pH of the composition is between 3.3 to 4.2.

In many aspects, typical buffering agents include acetate, glutamate, citrate, tartrate, benzoate, lactate, histidine or other amino acids, gluconate, phosphate and succinate. In certain aspects, the concentration of a buffering agent can be 0.1-100 mM. In other aspects, the buffering agent concentration is 1-50 mM. In yet other aspects, the buffering agent concentration is 2-3 mM.

In another aspect, the pharmaceutical compositions of the present disclosure are isotonic, i.e., in the range of 270-320 mOsm/kg. However, the compositions may have a tonicity in the range of 230-350 mOsm/kg. Therefore, the compositions may be either slightly hypotonic, 230-270 mOsm/kg, or slightly hypertonic, 320-350 mOsm/kg. In various embodiments, the tonicity of the pharmaceutical compositions is rendered isotonic by adjusting the concentration of any one or more of tonicity adjusting agent, co-solvent, buffering agent or the antioxidant in the solution.

In another aspect, the pharmaceutical compositions of the present disclosure may further comprise a tonicity agent. However, the compositions may further comprise multiple tonicity agents. Tonicity agents are well known in the art and commercially available. Useful tonicity agents include, for example, sodium chloride and dextrose.

The pharmaceutical compositions of the present disclosure are preferably packaged in pharmaceutically acceptable containers in many aspects. Pharmaceutically acceptable containers include intravenous bags, bottles, vials, and syringes. In certain embodiments, the containers include intravenous bags and syringes, which can be polymer-based, and vials and intravenous bottles, which can be made of glass. In some embodiments, the components of the container that come into contact with the pharmaceutical composition do not contain polyvinylchloride (PVC). In various aspects, the container is an intravenous bag that does not have any PVC containing components in contact with the pharmaceutical composition. It is also desirable to protect the pharmaceutical compositions from light. Therefore, the container may, optionally, further comprise a light barrier. In certain embodiments, the light barrier can be an aluminum over pouch.

In many aspects, the present disclosure also provides methods for preparing sterile pharmaceutical compositions. Examples of suitable procedures for producing sterile pharmaceutical drug products include, but are not limited to, terminal moist heat sterilization, ethylene oxide, radiation (i.e., gamma and electron beam), and aseptic processing techniques. Any one of these sterilization procedures can be used to produce the sterile pharmaceutical compositions described herein.

Sterile pharmaceutical compositions may also be prepared using aseptic processing techniques. Sterility is maintained by using sterile materials and a controlled working environment. All containers and apparatus are sterilized, preferably by heat sterilization, prior to filling. Then, the container is filled under aseptic conditions, such as by passing the composition through a filter and filling the units. Therefore, the compositions can be sterile filled into a container to avoid the heat stress of terminal sterilization.

The understanding of these and additional facets of the current application will be enhanced upon review of the following Examples. These examples aim to elucidate specific embodiments of the application, yet they do not seek to restrict its scope as delineated by the claims.

### EXPERIMENTAL RESULTS

A composition similar to RLD, Cardene® I.V. was prepared as a control and separately, a composition having a chelating agent and compositions with different antioxidants, viz., antioxidants acting by single electron transfer (SET) and antioxidants acting by Hydrogen atom transfer (HAT) were prepared as summarized in below Table 1. Further, comparative compositions containing different levels of a commonly used water-soluble HAT antioxidant, Ascorbic acid is provided in Table 2.

The above batches were manufactured in development laboratory conditions, wherein the laboratory was not sanitized using hydrogen peroxide to understand the impact of the chelating agent and different antioxidants on the formulation stability.

TABLE 1
Nicardipine hydrochloride compositions manufactured in development
laboratory area (without sanitization using Hydrogen peroxide)

| | | | Composition | | Composition containing | | | |
|---|---|---|---|---|---|---|---|---|
| | | | containing | | antioxidants acting by single | | | |
| | Control | | Chelating agent | | electron transfer (SET) | | | |
| | formulation, | | Composition 1: | | Composition 2: | | Composition 3: | |
| | similar to RLD, | | With 0.04 mg/mL | | With 1 mg/mL | | With 1 mg/mL | |
| Ingredients | Cardene ® IV | | Edetate disodium | | L-Cysteine HCl | | Monothioglycerol | |
| Nicardipine | 2.5 | mg | 2.5 | mg | 2.5 | mg | 2.5 | mg |
| Hydrochloride, USP | | | | | | | | |
| Sorbitol, NF | 48.0 | mg | 48.0 | mg | 48.0 | mg | 48.0 | mg |
| Citric Acid | 0.525 | mg | 0.525 | mg | 0.525 | mg | 0.525 | mg |
| Monohydrate, USP | | | | | | | | |
| Sodium Hydroxide, NF | 0.09 | mg | 0.09 | mg | 0.09 | mg | 0.09 | mg |

| | | | | | |
|---|---|---|---|---|---|
| Edetate disodium, USP | — | 0.04 | mg | — | — |

| | | | | | |
|---|---|---|---|---|---|
| L-Cysteine HCl, USP | — | — | 1.0 | mg | — |

| | | | | | |
|---|---|---|---|---|---|
| Monothioglycerol, NF | — | — | — | 1.0 | mg |

| | | | | |
|---|---|---|---|---|
| Sodium metabisulfite, NF | — | — | — | — |
| Ascorbic acid, USP | — | — | — | — |
| Butylated hydroxy | — | — | — | — |
| Anisole NF | | | | |
| Butylated hydroxy | — | — | — | — |
| Toluene NF | | | | |
| Citric Acid USP and/or | q.s to adjust pH | q.s to adjust pH | q.s to adjust pH | q.s to adjust pH |
| Sodium Hydroxide, NF* | to about 3.7 | to about 3.7 | to about 3.7 | to about 3.7 |
| Water for Injection, | q.s to 1 mL | q.s to 1 mL | q.s to 1 mL | q.s to 1 mL |
| USP | | | | |

| | | | Compositions containing | | | |
|---|---|---|---|---|---|---|
| | Composition containing | | antioxidants acting by | | | |
| | antioxidants acting by single | | Hydrogen atom transfer (HAT) | | | |
| | electron transfer (SET) | | | | Composition 6: | |
| | Composition 4: | | Composition 5: | | With 0.1 mg/mL | |
| | With 1 mg/mL | | With 1 mg/mL | | of BHA and | |
| Ingredients | Sodium metabisulfite | | Ascorbic acid | | 0.1 mg/L of BHT | |
| Nicardipine | 2.5 | mg | 2.5 | mg | 2.5 | mg |
| Hydrochloride, USP | | | | | | |
| Sorbitol, NF | 48.0 | mg | 48.0 | mg | 48.0 | mg |
| Citric Acid | 0.525 | mg | 0.525 | mg | 0.525 | mg |
| Monohydrate, USP | | | | | | |
| Sodium Hydroxide, NF | 0.09 | mg | 0.09 | mg | 0.09 | mg |

| Edetate disodium, USP | — | | — | — |
|---|---|---|---|---|
| L-Cysteine HCl, USP | — | | — | — |
| Monothioglycerol, NF | — | | — | — |
| Sodium metabisulfite, NF | 1.0 | mg | — | — |

| | | | | |
|---|---|---|---|---|
| Ascorbic acid, USP | — | 1.0 | mg | — |

| Butylated hydroxy | — | — | 0.1 | mg** |
|---|---|---|---|---|
| Anisole NF | | | | |
| Butylated hydroxy | — | — | 0.1 | mg** |
| Toluene NF | | | | |

| | | | |
|---|---|---|---|
| Citric Acid USP and/or | q.s to adjust pH | q.s to adjust pH | q.s to adjust pH |
| Sodium Hydroxide, NF* | to about 3.7 | to about 3.7 | to about 3.7 |
| Water for Injection, | q.s to 1 mL | q.s to 1 mL | q.s to 1 mL |
| USP | | | |

*Citric Acid Monohydrate, USP is used as 10% w/v solution and Sodium Hydroxide, NF is used as 0.1N solution for pH adjustments. These solutions are used for adjustment of pH of the solution to about 3.7 as required.
**The lipophilic antioxidants, BHA and BHT were pre-dissolved in appropriate quantity of ethanol and added to the composition.

TABLE 2
Nicardipine hydrochloride compositions containing various concentrations of a water-soluble HAT antioxidant,
Ascorbic acid manufactured in laboratory (without any sanitization using Hydrogen peroxide).

| | Composition 7: | | Composition 8: | | Composition 9: | | Composition 10: | | Composition 5: | |
|---|---|---|---|---|---|---|---|---|---|---|
| | With 0.05 mg/mL | | With 0.1 mg/mL | | With 0.25 mg/mL | | with 0.5 mg/mL | | With 1 mg/mL | |
| Ingredients | Ascorbic acid | | Ascorbic acid | | Ascorbic acid | | Ascorbic acid | | Ascorbic acid | |
| Nicardipine Hydrochloride, USP | 2.5 | mg | 2.5 | mg | 2.5 | mg | 2.5 | mg | 2.5 | mg |
| Sorbitol, NF | 48.0 | mg | 48.0 | mg | 48.0 | mg | 48.0 | mg | 48.0 | mg |
| Citric Acid Monohydrate, USP | 0.525 | mg | 0.525 | mg | 0.525 | mg | 0.525 | mg | 0.525 | mg |
| Sodium Hydroxide, NF | 0.09 | mg | 0.09 | mg | 0.09 | mg | 0.09 | mg | 0.09 | mg |
| Ascorbic acid, USP | 0.05mg | mg | 0.1 | mg | 0.25 | mg | 0.5 | mg | 1 | mg |

| | | | | | |
|---|---|---|---|---|---|
| Citric Acid Monohydrate, USP | q.s to adjust | q.s to adjust | q.s to adjust | q.s to adjust | q.s to adjust |
| and/or Sodium Hydroxide, NF* | pH to about 3.7 | pH to about 3.7 | pH to about 3.7 | pH to about 3.7 | pH to about 3.7 |
| Water for Injection, USP | qs to 1 mL | qs to 1 mL | qs to 1 mL | qs to 1 mL | qs to 1 mL |

*Citric Acid Monohydrate, USP is used as 10% w/v solution and Sodium Hydroxide, NF is used as 0.1N solution for pH adjustments. These solutions are used for adjustment of pH of the solution to about 3.7 as required.

### Example: Manufacturing Process

An example manufacturing process is outlined below:

Step 1: Water Collection and Cooling: Water for Injection, constituting approximately 95% of the batch size, was collected in a stainless steel compounding vessel and cooled to ambient temperature.

Step 2: Nitrogen Sparging: Nitrogen gas was used to sparge the Water for Injection, reducing dissolved oxygen levels to less than 1 ppm. Nitrogen sparging was maintained throughout the compounding process, with the exception of material addition stages to prevent powder dusting.

Step 3: Co-solvent/tonicity adjusting agent Addition: The batch quantity of sorbitol was added and mixed for 10 minutes until a clear solution was achieved.

Step 4: Buffering agent Addition: The batch quantity of citric acid monohydrate and sodium hydroxide was added and mixed for 10 minutes to obtain a clear solution.

Step 6: Drug substance Addition: The batch quantity of nicardipine hydrochloride was added and mixed for 45 minutes or until a clear solution was attained.

Step 7: Chelating agent and Antioxidant addition: The chelating agent and the antioxidants, as applicable to the compositions, were added and mixed for 10 minutes.

Step 8: pH Adjustment: The pH of the solution was verified, and adjustments were made, if necessary, using 10% citric acid solution and/or 0.1N sodium hydroxide solution to achieve a desired pH of approximately 3.7.

Step 9: Volume Adjustment: The batch volume was adjusted using pre-sparged cooled Water for Injection, USP.

Step 10: Final Verification: The final pH and dissolved oxygen content were confirmed after mixing for 5-10 minutes.

Step 11: Filtration: The product underwent filtration through 0.22 μm sterile filters.

Step 12: Vial Filling: The filtered bulk solution was filled into 10-mL Type I, Amber glass vials.

Step 13: Sealing: The filled units were stoppered and sealed under a reduced oxygen headspace of less than 10% oxygen.

The different formulations were evaluated for various critical quality attributes and further, the stability of the different compositions was evaluated at accelerated stability (40° C./75% RH) and long-term stability conditions.

The comparative stability of the different compositions containing antioxidants acting by single electron transfer (SET) are provided in Table 3. The comparative stability of the different compositions containing chelating agent and antioxidants acting by Hydrogen atom transfer (HAT) is provided in Table 4. The comparative stability of the compositions containing different concentrations of a water-soluble HAT antioxidant, Ascorbic acid is provided in Table 5.

TABLE 3
Comparative stability trend of control formulation and compositions
containing SET antioxidants (manufactured in development laboratory).

| | | | | | Compositions containing antioxidants | | |
|---|---|---|---|---|---|---|---|
| | | | | | acting by single electron transfer (SET) | | |
| | | Costrol formulation, | | | Composition 2: | | |
| | | (Similar to RLD, | | | With 1 mg of | | |
| | | Cardene ®) | | | L-Cysteine HCl | | |
| | | | 3 month, | 3 month, | | 3 month, | 3 month, |
| | | | 40° C./ | 25° C./ | | 40° C./ | 25° C./ |
| Parameters | Acceptance criteria | INITIAL | 75% RH | 60% RH | INITIAL | 75% RH | 60% RH |
| Description | A Clear yellow colour | A Clear, pale yellow | | | A Clear, pale yellow | | |
| | solution Essentially | colour solution | | | colour solution | | |
| | free from visible | | | | | | |
| | particles. | | | | | | |
| pH | 3.0-4.2 | 3.57 | 3.63 | 3.67 | 3.64 | 3.63 | 3.59 |
| Osmolality | 250 to 330 mOsm/kg | 291 | 288 | 285 | 291 | 286 | 288 |
| Assay | 90.0-110.0% | 101.6 | 96.9 | 98.2 | 93.6 | 90.9 | 90.9 |

Related substances by HPLC

| | | | | | | | |
|---|---|---|---|---|---|---|---|
| Nicardipine | NMT 2.5% | 0.12 | 0.16 | 0.21 | 2.38 | 3.89 | 4.24 |
| Pyridine | | | | | | | |
| Analog | | | | | | | |
| Nicardipine | NMT 0.2% | 0.01 | 0.11 | 0.06 | BDL | 0.09 | 0.06 |
| Mono Acid | | | | | | | |
| N-Benzyl-N- | NMT 0.7% | 0.03 | 0.62 | 0.25 | 0.06 | 0.62 | 0.30 |
| Methyl | | | | | | | |
| ethanolamine | | | | | | | |
| Total | NMT 3.5% | 0.24 | 1.39 | 0.59 | 2.57 | 5.18 | 4.43 |
| impurities | | | | | | | |

| | Compositions containing antioxidants | | | | | |
|---|---|---|---|---|---|---|
| | acting by single electron transfer (SET) | | | | | |
| | Composition 3: | | | Composition 4: | | |
| | With 1 mg of | | | With 1 mg of | | |
| | Monothioglycerol | | | sodium metabisulfite | | |
| | | 3 month, | 3 month, | | 3 month, | 3 month, |
| | | 40° C./ | 25° C./ | | 40° C./ | 25° C./ |
| Parameters | INITIAL | 75% RH | 60% RH | INITIAL | 75% RH | 60% RH |
| Description | A Clear, pale yellow | | | A Clear, pale yellow | | |
| | colour solution | | | colour solution | | |
| pH | 3.59 | 3.58 | 3.59 | 3.76 | 3.75 | 3.72 |
| Osmolality | 299 | 294 | 292 | 309 | 303 | 305 |
| Assay | 98.6 | 93.1 | 96.9 | 81.6 | 69.5 | 77.7 |
| Related substances by HPLC | | | | | | |
| Nicardipine | 3.50 | 3.81 | 4.23 | 1.49 | 9.29 | 6.28 |
| Pyridine | | | | | | |
| Analog | | | | | | |
| Nicardipine | BDL | 0.09 | 0.06 | BDL | 0.39 | 0.02 |
| Mono Acid | | | | | | |
| N-Benzyl-N- | 0.06 | 0.73 | 0.28 | 0.06 | 0.86 | 0.57 |
| Methyl | | | | | | |
| ethanolamine | | | | | | |
| Total | 3.96 | 5.21 | 4.70 | 4.45 | 21.27 | 12.61 |
| impurities | | | | | | |

Observations: The study indicates that the stability of the control formulation manufactured in development laboratory (wherein no sanitation using hydrogen peroxide is performed) is found satisfactory. However, in the formulations containing antioxidants acting by single electron transfer (SET), there was significant increase in the nicardipine pyridine analog level was observed. The observed level of Nicardipine pyridine analog and the total impurities exceeded the acceptance criteria recommended in USP monograph for Nicardipine Injection. The stability trend with SET antioxidants indicates the possibility that SET class of antioxidants may sensitize DHP compounds to form higher level of pyridine analog impurity.

TABLE 4
Comparative stability trend of control formulation and compositions containing
chelating agent and HAT antioxidants (Manufactured in development laboratory).

| | | | | | Composition containing | | |
|---|---|---|---|---|---|---|---|
| | | | | | chelating agent | | |
| | | Control formulation, | | | Composition 1: | | |
| | | (Similar to RLD, | | | With 0.04 mg/mL | | |
| | | Cardene ®) | | | Edetate disodium | | |
| | | | 3 month, | 3 month, | | 3 month, | 3 month, |
| | | | 40° C./ | 25° C./ | | 40° C./ | 25° C./ |
| Parameters | Acceptance criteria | INITIAL | 75% RH | 60% RH | INITIAL | 75% RH | 60% RH |
| Description | A Clear yellow colour | A Clear, pale yellow | | | A Clear, pale yellow | | |
| | solution Essentially | colour solution | | | colour solution | | |
| | free from visible | | | | | | |
| | particles. | | | | | | |
| pH | 3.0-4.2 | 3.57 | 3.63 | 3.67 | 3.42 | 3.42 | 3.41 |
| Osmolality | 250 to 330 mOsm/kg | 291 | 288 | 285 | 293 | 291 | 291 |
| Assay | 90.0-110.0% | 101.6 | 96.9 | 98.2 | 102.2 | 96.3 | 97.5 |

Related substances by HPLC

| | | | | | | | |
|---|---|---|---|---|---|---|---|
| Nicardipine | NMT 2.5% | 0.12 | 0.16 | 0.21 | 0.14 | 0.99 | 0.35 |
| Pyridine | | | | | | | |
| Analog | | | | | | | |
| Nicardipine | NMT 0.2% | 0.01 | 0.11 | 0.06 | BDL | 0.12 | 0.06 |
| Mono Acid | | | | | | | |
| N-Benzyl-N- | NMT 0.7% | 0.03 | 0.62 | 0.25 | 0.02 | 0.62 | 0.25 |
| Methyl | | | | | | | |
| ethanolamine | | | | | | | |
| Total | NMT 3.5% | 0.24 | 1.39 | 0.59 | 0.25 | 2.26 | 0.72 |
| impurities | | | | | | | |

| | Compositions containing antioxidants | | | | | |
|---|---|---|---|---|---|---|
| | acting by Hydrogen atom transfer (HAT) | | | | | |
| | Composition 5: | | | Composition 6: | | |
| | With 1 mg/mL | | | With 0.1 mg/mL of BHA | | |
| | Ascorbic acid | | | and 0.1 mg/mL of BHT | | |
| | | 3 month, | 3 month, | | 3 month, | 3 month, |
| | | 40° C./ | 25° C./ | | 40° C./ | 25° C./ |
| Parameters | INITIAL | 75% RH | 60% RH | INITIAL | 75% RH | 60% RH |
| Description | A Clear, pale yellow | | | A Clear, pale yellow | | |
| | colour solution | | | colour solution | | |
| pH | 3.53 | 3.59 | 3.58 | 3.52 | 3.56 | 3.58 |
| Osmolality | 292 | 290 | 290 | 486 | 483 | 494 |
| Assay | 101.4 | 94.7 | 96.5 | 101.5 | 95.9 | 97.3 |
| Related substances by HPLC | | | | | | |
| Nicardipine | 0.11 | 0.21 | 0.17 | 0.15 | 0.69 | 0.19 |
| Pyridine | | | | | | |
| Analog | | | | | | |
| Nicardipine | BDL | 0.12 | 0.08 | 0.01 | 0.10 | 0.05 |
| Mono Acid | | | | | | |
| N-Benzyl-N- | 0.05 | 0.68 | 0.31 | 0.06 | 0.57 | 0.23 |
| Methyl | | | | | | |
| ethanolamine | | | | | | |
| Total | 0.32 | 1.74 | 0.80 | 0.92 | 2.23 | 0.91 |
| impurities | | | | | | |

Observations: The study indicates that the stability of the formulation having antioxidants acting by Hydrogen atom transfer (HAT) is similar to control formulation. There was no significant increase in the Nicardipine pyridine analog impurity level was seen over the stability studies. Compared to the formulation containing the chelating agent, the level of Pyridine analog was found lower in the formulation containing Ascorbic acid as antioxidant. Further, the osmolality of the formulation containing lipophilic antioxidant, BHA/BHT was found higher than the desired level of 270 mOsm/kg to 330 mOsm/kg as ethanol was required to dissolve the BHT/BHT.

TABLE 5
Comparative stability trend of compositions containing different concentrations of a water-
soluble HAT antioxidant, Ascorbic acid (Manufactured in development laboratory).

| | | Composition 7: | | | Composition 8: | | |
|---|---|---|---|---|---|---|---|
| | | With 0.05 mg/mL | | | With 0. 1 mg/mL | | |
| | | Ascorbic acid | | | Ascorbic acid | | |
| | | | 3 month, | 3 month, | | 3 month, | 3 month, |
| | | | 40° C./ | 25° C./ | | 40° C./ | 25° C./ |
| Parameters | Acceptance criteria | INITIAL | 75% RH | 60% RH | INITIAL | 75% RH | 60% RH |
| Description | A Clear yellow colour | A Clear, pale yellow | | | A Clear, pale yellow | | |
| | solution Essentially | colour solution | | | colour solution | | |
| | free from visible | | | | | | |
| | particles. | | | | | | |
| pH | 3.0-4.2 | 3.53 | 3.59 | 3.57 | 3.49 | 3.51 | 3.53 |
| Osmolality | 250 to 330 mOsm/kg | 295 | 294 | 292 | 293 | 290 | 290 |
| Assay | 90.0-110.0% | 102.4 | 96.6 | 97.4 | 100.7 | 95.1 | 97.6 |

Related substances by HPLC

| | | | | | | | |
|---|---|---|---|---|---|---|---|
| Nicardipine | NMT 2.5% | 0.21 | 0.29 | 0.32 | 0.21 | 0.37 | 0.26 |
| Pyridine | | | | | | | |
| Analog | | | | | | | |
| Nicardipine | NMT 0.2% | BDL | 0.12 | 0.08 | BDL | 0.14 | 0.08 |
| Mono Acid | | | | | | | |
| N-Benzyl-N- | NMT 0.7% | 0.04 | 0.64 | 0.31 | 0.07 | 0.76 | 0.32 |
| Methyl | | | | | | | |
| ethanolamine | | | | | | | |
| Total | NMT 3.5% | 0.39 | 1.66 | 0.85 | 0.45 | 2.47 | 0.47 |
| impurities | | | | | | | |

| | Composition 9: | | | Composition 10: | | |
|---|---|---|---|---|---|---|
| | With 0.25 mg/mL | | | With 0.5 mg/mL | | |
| | Ascorbic acid | | | Ascorbic acid | | |
| | | 3 month, | 3 month, | | 3 month, | 3 month, |
| | | 40° C./ | 25° C./ | | 40° C./ | 25° C./ |
| Parameters | INITIAL | 75% RH | 60% RH | INITIAL | 75% RH | 60% RH |
| Description | A Clear, pale yellow | | | A Clear, pale yellow | | |
| | colour solution | | | colour solution | | |
| pH | 3.55 | 3.61 | 3.59 | 3.56 | 3.59 | 3.57 |
| Osmolality | 293 | 292 | 292 | 288 | 289 | 289 |
| Assay | 102.3 | 95.9 | 97.8 | 101.4 | 94.3 | 95.9 |
| Related substances by HPLC | | | | | | |
| Nicardipine | 0.16 | 0.26 | 0.23 | 0.12 | 0.22 | 0.18 |
| Pyridine | | | | | | |
| Analog | | | | | | |
| Nicardipine | BDL | 0.12 | 0.08 | BDL | 0.12 | 0.08 |
| Mono Acid | | | | | | |
| N-Benzyl-N- | 0.05 | 0.70 | 0.31 | 0.12 | 0.68 | 0.30 |
| Methyl | | | | | | |
| ethanolamine | | | | | | |
| Total | 0.39 | 1.78 | 0.82 | 0.34 | 1.69 | 0.75 |
| impurities | | | | | | |

Observations: The study indicates that the stability of the formulation having different concentrations of a water-soluble HAT antioxidant, Ascorbic acid is found satisfactory and comparable. There was no significant increase in the Nicardipine pyridine analog impurity level was seen in the stability assessment in the concentration as low as 0.05 mg/mL of Ascorbic acid.

Though the level of DHP Pyridine analog impurity did not increase significantly in the control formulation (composition similar to Cardene® I.V) when manufactured in development laboratory conditions, the level of this impurity increases significantly when the product was manufactured aseptically under commercial/production scale manufacturing process conditions.

The comparative stability profile of the control formulation, similar to RLD, Cardene® which does not contain any antioxidant and the formulation containing water-soluble HAT antioxidant, Ascorbic acid manufactured in the development laboratory and in a commercial scale production area is provided in Table 6.

TABLE 6
Comparative stability of control formulation and the formulation containing water-soluble HAT antioxidant
(Ascorbic acid) manufactured in the development laboratory and in a commercial scale production area.

| | | Control formulation, | | | Control formulation, | |
|---|---|---|---|---|---|---|
| | | (Similar to RLD, | | | (Similar to RLD, | |
| | | Cardene ®) manufactured in | | | Cardene ®) manufactured in | |
| | | Development laboratory | | | manufacturing/production area | |
| | | | 3 month, | 3 month, | | 1 month, |
| | | | 40° C./ | 25° C./ | | 40° C./ |
| Parameters | Acceptance criteria | INITIAL | 75% RH | 60% RH | INITIAL | 75% RH |
| Description | A Clear yellow colour | A Clear, pale yellow | | | A Clear, pale yellow | |
| | solution Essentially free | colour solution | | | colour solution | |
| | from visible particles. | | | | | |
| pH | 3.0-4.2 | 3.57 | 3.63 | 3.67 | 3.42 | 3.69 |
| Osmolality | 250 to 330 mOsm/kg | 291 | 288 | 285 | 290 | NP* |
| Assay | 90.0-110.0% | 101.6 | 96.9 | 98.2 | 99.1 | 97.2 |

Related substances by HPLC

| | | | | | | |
|---|---|---|---|---|---|---|
| Nicardipine | NMT 2.5% | 0.12 | 0.16 | 0.21 | 0.03 | 3.51 |
| Pyridine Analog | | | | | | |
| Nicardipine | NMT 0.2% | 0.01 | 0.11 | 0.06 | BDL | 0.05 |
| Mono Acid | | | | | | |
| N-Benzyl-N- | NMT 0.7% | 0.03 | 0.62 | 0.25 | BDL | 0.24 |
| Methyl | | | | | | |
| ethanolamine | | | | | | |
| Total impurities | NMT 3.5% | 0.24 | 1.39 | 0.59 | BQL | 3.87 |

| | Composition containing | | | Composition containing | |
|---|---|---|---|---|---|
| | 1 mg/mL Water-soluble HAT | | | 1 mg/mL Water-soluble HAT | |
| | antioxidant, Ascorbic acid | | | antioxidant, Ascorbic acid | |
| | manufactured in | | | manufacturing/ | |
| | Development laboratory | | | production area | |
| | | 3 month, | 3 month, | | 1 month, |
| | | 40° C./ | 25° C./ | | 40° C./ |
| Parameters | INITIAL | 75% RH | 60% RH | INITIAL | 75% RH |
| Description | A Clear, pale yellow | | | A Clear, pale yellow | |
| | colour solution | | | colour solution | |
| pH | 3.53 | 3.59 | 3.58 | 3.42 | 3.37 |
| Osmolality | 292 | 290 | 290 | 296 | NP* |
| Assay | 101.4 | 94.7 | 96.5 | 98.3 | 97.4 |
| Related substances by HPLC | | | | | |
| Nicardipine | 0.11 | 0.21 | 0.17 | 0.01 | 0.11 |
| Pyridine Analog | | | | | |
| Nicardipine | BDL | 0.12 | 0.08 | BDL | 0.05 |
| Mono Acid | | | | | |
| N-Benzyl-N- | 0.05 | 0.68 | 0.31 | BDL | 0.25 |
| Methyl | | | | | |
| ethanolamine | | | | | |
| Total impurities | 0.32 | 1.74 | 0.80 | BQL | 0.47 |

*Test not performed during stability as Osmolality is not expected to change over the stability.

Observations: The stability data indicates that in the control formulation, (similar to RLD, Cardene®), the pyridine analog impurity increased significantly in the aseptically filled vials manufactured in the commercial production area within 1 month accelerated stability period. The root cause for this abnormal degradation behavior between the batches manufactured under the R&D laboratory conditions and commercial production/manufacturing conditions is not clearly understood. However, it appears to be related to the residues of hydrogen peroxide that may remain in the area after the area sanitization or the residues from the sterilization indicators used in the autoclaving of components, leading to the sensitization/degradation of DHP compounds.

In comparison to control formulation and the formulations containing antioxidants acting by single electron transfer (SET), it was surprisingly observed that the formulations containing HAT antioxidants possess significantly improved stability wherein Pyridine analog impurity levels are controlled well within the acceptance criteria irrespective of whether the batches are manufactured in R&D laboratory area or the production/manufacturing area.

The present invention offers several advantages. The pharmaceutical composition provides improved stability of 1,4-Dihydropyridines (DHP) due to the presence of hydrogen atom transfer (HAT) antioxidants. These HAT antioxidants effectively prevent the sensitization of DHP compounds to degrade to pyridine analog impurities by the residues of sanitizing agents that may remain in the area after the area sanitization or the volatile residues from sterilization indicators that are used in the autoclaving of components, ensuring consistency and integrity of the product over time. By incorporating HAT antioxidants, the composition mitigates the degradation of DHP compounds, thereby prolonging the shelf-life and maintaining the potency of the pharmaceutical product.

The formulation allows for flexibility in adjusting the concentration of nicardipine hydrochloride, HAT antioxidants, co-solvents, buffering agents, and tonicity agents, providing versatility in meeting specific formulation requirements and optimizing therapeutic outcomes. The pharmaceutical composition is designed for case of administration, whether through intravenous injection or other appropriate routes, ensuring patient convenience and compliance with treatment regimens. Despite challenges observed in commercial-scale manufacturing environments, the formulation's stability can be maintained with proper incorporation of antioxidants, enabling efficient production and distribution of the pharmaceutical product on a larger scale.

It will be understood by those skilled in the art that the foregoing general description and the following detailed description are exemplary and explanatory of the disclosure and are not intended to be restrictive thereof. While specific language has been used to describe the disclosure, any limitations arising on account of the same are not intended.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all the acts need to be necessarily performed. Also, those acts that are not dependent on other acts may be performed in parallel with the other acts. The scope of embodiments is by no means limited by these specific examples.

We claim:

1. A stabilized, pharmaceutical aqueous composition of 1,4-Dihydropyridines with hydrogen atom transfer (HAT) antioxidants, wherein the composition comprises:
a pharmaceutically acceptable salt of 1,4-Dihydropyridines (DHP), comprising nicardipine hydrochloride in an amount of 0.1 mg/mL to 5.0 mg/mL;
atleast one hydrogen atom transfer (HAT) antioxidant stabilizer in an amount of 0.001 mg/mL to 25 mg/mL;
wherein the stabilized composition comprises a pyridine analog impurity in an amount less than 1% w/w of the DHP compound.

2. The pharmaceutical composition as claimed in claim 1, wherein hydrogen atom transfer (HAT) antioxidant is selected from hydrophilic or lipophilic compounds comprising ascorbic acid, gallic acid, caffeic acid, ferulic acid, coumaric acid, Butylated hydroxyanisole (BHA), Butylated hydroxytoluene (BHT), or derivatives thereof.

3. The pharmaceutical composition as claimed in claim 1, further comprises a co-solvent selected from sorbitol, mannitol, xylitol, propylene glycol, polyethylene glycol, ethanol, water, or a combination thereof, in an amount of 0.1 mg/mL to 50 mg/mL of the composition.

4. The pharmaceutical composition as claimed in claim 1, further comprising a buffering agent selected from citric acid, anhydrous citric acid, citric acid monohydrate, sodium hydroxide, acetate, glutamate, citrate, tartrate, benzoate, lactate, histidine, amino acids, gluconate, phosphate, succinate, or a combination thereof, in an amount of 0.1 mg/mL to 50 mg/mL of the composition, wherein the pH is adjusted between 3 to 5.

5. The pharmaceutical composition as claimed in claim 1, further comprising a tonicity agent selected from sodium chloride, dextrose, sucrose, xylitol, fructose, glycerol, sorbitol, mannitol, or sodium chloride to maintain an osmolality between 250 mOsm/kg to 350 mOsm/kg.

6. A pharmaceutical composition comprising a 1,4-dihydropyridine compound comprising nicardipine in an amount of 2.5 mg/mL of the composition and atleast one antioxidant acting by Hydrogen atom transfer (HAT) in an amount of 0.001 mg/mL to 25 mg/mL, wherein the HAT antioxidant is preferably Ascorbic acid or its pharmaceutically acceptable salts thereof.

7. A stabilized pharmaceutical composition comprising nicardipine hydrochloride in an amount of 1 mg/mL to 10 mg/mL, a co-solvent comprising sorbitol in an amount that is 40 mg/mL to 55 mg/mL of the composition, a buffering agent comprising citrate buffer in an amount between that is 0.1 mg/mL to 5 mg/mL of the composition, and atleast one antioxidant acting by Hydrogen atom transfer (HAT) in an amount of 0.001 mg/mL to 25 mg/mL, wherein the HAT antioxidant is preferably Ascorbic acid or its pharmaceutically acceptable salts thereof, wherein the stabilized composition comprises a pyridine analog impurity in an amount less than 1% w/w of the DHP compound.
